(19) United States
(12) Patent Application Publication
(10) Pub. No.: US 2011/0072978 A1
(43) Pub. Date: Mar. 31, 2011

(54) Method and Apparatus for Determining Taste Degradation of Coffee under Thermal Load

(76) Inventor: Bogdan Popescu, Redmond, WA (US)

(21) Appl. No.: 12/890,629

(22) Filed: Sep. 25, 2010

Related U.S. Application Data
(60) Provisional application No. 61/277,491, filed on Sep. 26, 2009.

(51) Int. Cl. G01N 33/14; A47J 31/44; B65D 81/38; A47G 19/14; B67D 7/06; G01K 13/00

(52) U.S. Cl. 99/288; 220/592.2; 222/475.1; 222/23; 702/130; 73/53.01

(57) ABSTRACT

Methods and apparatus for calculating and displaying the cumulated degradation of taste in coffee or tea based on monitored temperature history are disclosed. The degradation of taste display apparatus includes an immersible or wall-mounted temperature sensor and a means for calculating TBDS (total burn damage score) based on formulae configured for predicting changes in flavor substances. Based on the same method of calculating taste loss during a thermal exposure, a heating method and improved apparatus for heating coffee or tea in a serving vessel is also disclosed. The heating method uses induction heating together with a timer or temperature control and is configured for desktop, table, or car uses.

### PRIORITY DOCUMENTS

This application claims the benefit of 35 USC 119(e), claiming priority to U.S. Provisional Patent Application No. 61/277,491 filed 26 Sep. 2009, which is incorporated herein in entirety by reference.

### FIELD OF THE INVENTION

This invention is related to methods and apparatus for determining the degradation in taste of coffee as a function of temperature as well as ways of showing it, and a derived method of heating that would minimize the loss of taste.

### BACKGROUND

Coffee is an extremely popular beverage, and according to some reports, as many as 300 million cups of coffee are consumed per day in the United States alone. While much has been done to optimize the initial brewing of coffee, the problem of optimizing the flavor of coffee during an extended period after brewing has not been adequately addressed. The most frequent complaints are that the coffee is too hot or too cold, that it develops a “burnt” taste over time, or that desirable flavor notes are lost.

The main factors affecting coffee satisfaction after the first 20 minutes or more are that it either became too cold or, especially if it has been kept at elevated temperatures, that it starts acquiring a bitterly, unpleasant taste, many times referred at as “burnt taste”.

The standards have evolved over time. The National Coffee Association of USA, Inc has recommended coffee be maintained between 180 and 185° F. for optimal taste. These benefits have been reconsidered especially after a widely reported incident at a corporate vendor's where coffee was spilled into a customer's lap at a range of 195° F. to 205° F., a temperature at which serious tissue burns occur and at which flavor is expected to degrade rapidly.

On the other hand, other studies showed that for a large population of consumers the preferred temperature range is much lower than industry recommendations, and is instead about 139.6±14.8° F. (Lee and O'Mahony-2002, J Food Sci 67:2774-77) or about 125 and 155 degrees Fahrenheit. Another study shows the optimal temperature at about 136° F. (Brown and Diller-2008, Burns 34:648-54).

An 8-12 fl oz cup of coffee for example, consumed by sipping, where each sip has a volume of 0.1 to 0.5 fl oz, could be consumed over a period of more than an hour with intermittent sipping. It is not uncommon for somebody to prolong sipping from a cup of coffee for more than an hour, only to discover the coffee is cold and less satisfying. That same consumer, using a heating plate such as “Coffee Cup Warmer” (available commercially from Brookstone, Merrimack N.H., which relies on a preset level of resistive heating to plateau at about 120° F.), soon discovers that the coffee is kept warm but the taste has changed. The same happens when a carafe is placed on the heating plate of a coffee maker. The contents inevitably acquire a burnt or bitter aftertaste.

There are a number of methods to reheat coffee to bring it to a desirable temperature. Perhaps the oldest method for reheating coffee was to pour it back in the pot and set it over the fire. The method almost exclusively used currently for on location heating relies on resistive elements to heat a hot plate on which a cup or coffee pot is placed. However, the insulative properties of a ceramic cup or the glass of the coffee pot work against the device which leads to a very large heating time. Overheating becomes an additional problem when the liquid volume remaining in the cup is reduced.

Alternatively, coffee may be reheated by microwaving. This method provides fast heating directly to the coffee since the electromagnetic field penetrates the wall of a ceramic or glass mug. However, microwave magnetrons are noisy, need cooling, and require to be enclosed in bulky metal housings and a door to contain the electromagnetic field.

Another method, yet to be developed, would combine the versatility of the resistive element with the speed of the microwave oven is the electromagnetic induction heating as described in U.S. patent application Ser. No. 12/493,077. According to industry figures, induction heating efficiency is about 90%, as compared to 40% for gas burners and 47% for electric ranges. The application discloses a desktop inductive heater for heating a beverage in a ceramic cup, where the inductively responsive heating “cartridge” is disengageably inserted inside the cup or vessel.

It will be shown that choosing the right heating method can make a significant difference in the temperature-taste performance.

The coffee chemistry is very complicated. There are many chemical reactions that contribute to the taste, from brewing, which extracts the oils and essences, to reactions that happen when coffee is just sitting in an open cup. For example, chemical reactions with oxygen degrade flavor and are also accelerated by heating. Oxidative reactions with heating cause rapid “ageing” of the coffee constituents, as is also true of infusion beverages in general, such as tea, which is also widely consumed.

At this time, there is no way to know how much taste degradation to expect before drinking from the cup. Thus, there is a need in the art, for a method to differentiate between different types of heating in order to overcome the above disadvantages and to permit coffee to be enjoyed for an extended period of time near an optimal temperature without undesirable changes in flavor. Complementary to this need is an apparatus for assessing the potability of coffee or tea in a vessel and alerting the user if the flavor is expected to have deteriorated to an unacceptable level. Since satisfaction in drinking a hot beverage is a psychophysiological value, and depends on both temperature and flavor, mere measurement of temperature is insufficient to adequately predict consumer reaction. The prior art is silent on the problem of quantitatively predicting satisfaction in a hot beverage in real time at the point of use. The present invention addresses these problems as known in the art of hot beverages, particularly coffee and tea, and more generally, addresses the dependence of taste degradation on temperature history.

### SUMMARY

As discussed above, optimal coffee serving conditions present a paradox where service is extended for more than about 20 minutes following brewing—coffee that has become lukewarm on standing is undesirable; coffee that remains hot but has acquired burnt overtones or lost flavor is equally undesirable. A solution to this problem has the potential to bring satisfaction to many increasingly sophisticated consumers who daily return to their desk, workstation or favorite window nook with a cup of coffee in hand.

Disclosed is a method and apparatus for determining taste degradation or, generally, a change in taste in beverages like coffee or tea exposed to a temperature history. The method provides a taste degradation score at any point in time and is a tool to discriminate between different reheating methods.

Also disclosed is a method and apparatus for preserving coffee flavors while still enjoying a warm cup of coffee by letting the coffee in the cup, carafe, or other vessel cool between tastings and quickly bringing coffee to a preferred temperature for consumption precisely at the time of tasting.

The taste degradation assessment method is based on the realization that the “burnt” taste of coffee or tea is a consequence of chemical reactions that take place in the coffee mix. Since the rate of chemical reactions is affected by temperature so is the taste

It will be shown that, based on analytical relations and empirical observations, it is possible to quantitatively estimate changes in taste for a beverage containing temperature sensitive ingredients when exposed to a known temperature profile. Having this information allows the consumer to determine whether to continue to drink from the vessel or to prepare a fresh batch without having to taste the contents. Many of us have grown fuzz on our tongues from tasting vilely bad coffee from a pot that sat on a hot plate for just a few hours, for example. A device for recording the cumulative burn damage of a beverage from its temperature history, surprisingly, is fully effective in assessing the condition of a hot beverage and preventing a repeat of this undesirable experience as well as helping decide ahead of time when to brew new fresh coffee.

The invention also relates to devices for minimizing thermal damage when coffee temperature needs to be maintained. Currently available appliances to accomplish this are either thermally insulated vessels intended to passively delay cooling or act by continuously heating the vessel, and hence the liquid.

Instead, an inventive control model is proposed where liquid coffee is cooled when not in use and reheated rapidly just at the time when it is to be consumed. In this ‘on demand’ heating model, the flavors of the coffee are brought up to the preferred temperature for consumption only for brief periods and are otherwise preserved by allowing the cup and contents to cool when not in use. In this way, the cumulative temperature exposure of each particular cup of coffee is modulated at the point of use so that flavor deterioration is avoided. For this to be practical, an apparatus that has a small profile (so that it can be used on a desk), be able to heat coffee quickly, produce little or no noise, and consume modest power is required.

Induction heating has a much faster response for heating small volumes of liquid than through vessel resistive heating. In a preferred embodiment, the liquid is reheated by direct contact with an inductive element, bypassing layers of resistance to thermal diffusion across the walls of the vessel. As described in my co-pending U.S. patent application Ser. No. 12/493,077, a ferromagnetic disk placed inside a ceramic cup responds instantaneously to an inductive field, allowing for much faster intermittent reheating.

Using inductive reheating, a method for desktop appliance rewarming of coffee is disclosed wherein the temperature history of the coffee is purposefully kept low. The coffee is reheated only “on demand” at the time of use and is otherwise allowed to cool. Bursts of inductive energy are emitted in the liquid in response to a user “make ready” command so that thermal damage is minimized. Thermal overshoot may be avoided by judicious design of the energy budget done by the user or by use of sensors with feedback control.

In one embodiment, the desktop appliance for rewarming a ceramic coffee cup engaged thereon includes an inductive coil controlled by a button or other actuator, where the button activates a heating cycle, and the user has only to press the button to rewarm the coffee in a matter of tens of seconds.

Heating is discontinued during periods where no user instruction is provided. By allowing the coffee to cool during a “quiescent period” of standing, more flavor is preserved without sacrifice of thermal comfort when needed. This method is also useful with coffee made from “pods” as are currently popular, and with coffee brewed by percolation of grounds or instant coffee more generally, including expresso, café au lait, chocolate mocha, chicory blends, and other coffees with complex flavoring, and also teas.

The invention is realized in methods and apparatus for preserving flavors of a cup of coffee based on mathematical models showing the relationship between temperature history and flavor. The calculation formula provides a method for numerically expressing burn damage to a coffee beverage as a result of cumulative exposure to elevated temperatures or to extreme temperatures during its “life” in the cup. A correlation can be established between the burn index and taste so that predicted satisfaction can be numerically expressed (i.e., quantified).

Based on this approach, an apparatus is presented that in addition to temperature information also displays a cumulative taste degradation score. This taste degradation calculator can take the form of an enhanced thermometer that can be attached on the wall of the coffee mug or it can be embedded in the body of a traveling mug or coffee carafe. It can also be part of a coffee heating unit.

The foregoing and other objectives, features, and advantages of the invention will be more readily understood upon consideration of the following detailed description of the invention, taken in conjunction with the accompanying drawings.

### BRIEF DESCRIPTION OF THE DRAWINGS

The teachings of the present invention are more readily understood by considering the drawings, in which:

FIG. 1 is a plot illustrating the exponential loss of heat from a liquid in a ceramic coffee mug, where the initial temperature is about 130° F.

FIG. 2 plots the incremental increase in temperature of a liquid in a ceramic coffee mug when heated with a resistive heating “desktop warmer” of the prior art.

FIG. 3 presents the cooling of a quantity of liquid in an open ceramic mug (dotted line) as compared to a thermally insulated travel mug (solid line).

FIG. 4 shows typical chemical reaction rates as a function of temperature for different values of the activation energy (20 kJ/mol; 90 kJ/mol; 150 kJ/mol).

FIG. 5 plots a burn damage rate as a function of temperature using a discontinuous, stepwise function.

FIG. 6A shows two resistive heating patterns. For the first, the coffee is heated to the same high temperature and held at constant temperature on a hot plate. For the other pattern, coffee is repetitively cycled thru heating to 112° F. and natural cooling down to 90° F., forming a sawtooth wave in the heating profile. The first pattern is representative of existing cup warmers with setpoint power control. Using an integral of the burn damage rate (BD), a total burn damage score (TBDS) for the two cases at 1, 2 and 3 hrs is given in FIG. 6B.

FIG. 7A represents a modified resistive pattern where the final temperature reaches 124° F. when the coffee is progressively consumed, factoring decreasing volume over time into the thermal profile. Again constant resistive heating and intermittent inductive heating are compared. FIG. 7B shows the total burn damage scores (TBDS) for the two patterns.

FIGS. 8A and 8B compare heating patterns and burn damage rate for resistive heating and thermal insulation.

FIGS. 9A and 9B compare thermal histories and burn damage rate for inductive heating and thermally insulated travel mugs, where modulated inductive heating mimics the natural cooling profile for the thermally insulated travel mug.

FIG. 10A models a thermal history for an inductive reheating device which operates on a 45 minute periodic cycle with a target setpoint, assuming a progressively smaller volume as the coffee is consumed. FIG. 10B plots the resultant burn damage as TBDS for 1, 2 and 3 hrs.

FIG. 11 shows an induction heating coffee cup warmer unit with an incremental timer and TBDS display for displaying burn damage. A mug is shown for reference as would be seated on the warmer unit.

FIG. 12 schematically represents a cutaway view of a combination of a cup, internal cartridge with RFID tag with temperature sensor, and an inductive heating unit.

FIG. 13 illustrates an insertable probe with TBDS display for displaying burn damage clipped to a mug.

FIG. 14 is a travel mug with internal temperature sensor and TBDS burn damage indicator.

FIG. 15 is a coffee carafe with internal temperature sensor and TBDS burn damage indicator.

FIG. 16 indicates a logic path for continuously incrementing a burn damage rate (BD) as a function of the updated temperature history and displaying a TBDS value, where TBDS is a cumulative total burn damage score.

### NOTATION AND NOMENCLATURE

Certain terms throughout the following description are used to refer to particular features, steps or components, and are used as terms of description and not of limitation. As one skilled in the art will appreciate, different persons may refer to the same feature, step or component by different names. Components, steps or features that differ in name but not in function or action are considered equivalent and not functionally distinguishable, and may be substituted herein without departure from the invention. Certain meanings are defined here as intended by the inventor, i.e. they are intrinsic meanings. Other words and phrases used here take their meaning as consistent with usage as would be apparent to one skilled in the relevant arts.

“Burn damage rate” (BD)—refers to the derivative of burn damage with temperature, i.e. d(BD)/dT. Since the temperature is a function of time, the “burn damage rate” also becomes a function of time.

Total burn damage score (TBDS)—refers to the integral of the cumulated burn damage rate over a duration of time.

“Thermal burn damage monitor”—refers to a temperature sensor unit with functionality for on-board calculation of the burn damage rate and the total burn damage score.

“Temperature history”—refers to the beverage temperature variation over the entire time from the moment monitoring is initiated to the point of measurement and up to complete consumption. The temperature history may be viewed as a profile or plot of temperature versus time.

“Beverage”—a potable aqueous liquid. Of particular interest in the practice of the present invention are beverages known for delicate flavors that decay over time after the beverage is brewed. These beverages are often infusions of plant materials such as coffees or teas.

“Comfort zone”—a range of temperatures characterized subjectively as not too hot and not too cold by a consumer of a beverage.

“Exponential decay”—rapid decay resembling an exponential function, but not necessarily in strict mathematical sense.

“Inductive heating”—relates to heating by electrical induction, where an oscillating magnetic field heats an inductively responsive material by induction of eddy currents and, in case of ferromagnetic materials, by a combination of eddy currents and magnetic hysteresis.

“Inductively heatable materials”—materials in which significant electrical current is induced when said material is subjected to a changing magnetic field, currents which, by the Joule effect, produce heat; i.e. materials that are responsive to an oscillating magnetic field and dissipate the power of the field by generating caloric heat. These materials include without limitation iron, cast iron, steel, carbon steel, and some stainless steel alloys. Aluminum and copper and their alloys are responsive to magnetic fields but their use is not practicable with the majority of currently available inductive heating appliances.

“Insertable”—able to be put into something else, as in an “insertable cartridge”, where the cartridge is inserted into the interior cavity of a vessel.

“Cartridge” or “puck”—an insertable member or layer of an inductively responsive material formed as a body having a shape and stiffness adapted for handling and for insertion into the inside cavity of a cup or vessel.

“Vessel”—includes cups etc, insulated travel mugs, carafes, percolator pots, and so forth. A “vessel” is an article generally for preparation of or for containing a beverage, having a peripheral wall, a lip, a generally flat bottom with external base, and an internal or inside cavity, where the inside cavity is generally accessible through an opening at the top of the vessel.

A “method”—as disclosed herein refers one or more steps or actions for achieving the described end. Unless a specific order of steps or actions is required for proper operation of the embodiment, the order and/or use of specific steps and/or actions may be modified without departing from the scope of the present invention.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, particular features, structures, or characteristics of the invention may be combined in any suitable manner in one or more embodiments.

“Conventional”—refers to a term or method designating that which is known and commonly understood in the technology to which this invention relates.

Unless the context requires otherwise, throughout the specification and claims which follow, the word “comprise” and variations thereof, such as, “comprises” and “comprising” are to be construed in an open, inclusive sense, that is as “including but not limited to”.

The appended claims are not to be interpreted as including means-plus-function limitations, unless such a limitation is explicitly recited in a given claim using the phrase “means for.”

### DETAILED DESCRIPTION

Although the following detailed description contains many specific details for the purposes of illustration, one of skill in the art will appreciate that many variations and alterations to the following details are within the scope of the invention. Accordingly, the exemplary embodiments of the invention described below are set forth without any loss of generality to, and without imposing limitations upon, the claimed invention.

Since the “burnt” taste of a plant infusion such as coffee, which is a complex organic mixture, is hard to measure, some assumptions are based on sensorial perception and evaluation rather than precise measurements. However, these assumptions are captured in a mathematical model which is intended to preserve the main characteristics of the physical phenomenon.

A correlation may be established between a Total burn damage score (TBDS) and taste so that “satisfaction” can be numerically expressed (i.e., quantified) and predicted. The calculation by a choice of formulas provides a method for numerically expressing burn damage to a coffee beverage as a result of cumulative exposure to elevated temperatures or to extreme temperatures during the “life” of the beverage in the cup or vessel.

While not generally recognized, changes in taste can be the effect of chemical reactions and changes in reactant concentration. It will be assumed here that one or more types of chemical reactions are responsible for the acquired burnt coffee taste. Moreover, it is known that chemical reactions are affected by temperature. Even though the chemistry of coffee can be very complicated, a generic variation of chemical reaction rate with temperature is assumed for modeling.

A well known relation for chemical reaction rate dependency on temperature is the so-called Arrhenius equation. The rate of chemical reactions is given according to the formula

k=Ae^{-Ea/RT} (Eq 1)

where T is temperature measured in ° K, R is the gas constant, Eₐ is an activation energy, and A is a proportionality constant.

There are also modified forms of this equation. One form makes explicit the temperature dependence of the pre-exponential factor:

k=A(T/T₀)ⁿe^{-Ea/RT} (Eq 2)

where T₀ is a reference temperature and allows n to be a unitless power with usual values between −1 and 1. It reverts to equation (1) when n=0.

Another form of the equation is the stretched exponential form:

$$k = A {e}^{[ - \frac{{Ea}^{β}}{RT} ]} \qquad ( Eq 3 )$$

where β is a unitless number of order 1 which can either have a theoretical meaning or can be chosen to better fit experimental data.

Another form of the equation, called the Eyring-Polanyi equation, is:

$$k = \frac{{k}_{B} T}{ℏ} {e}^{- [ \frac{Δ {G}^{‡}}{RT} ]} \qquad ( Eq 4 )$$

where ΔG^{‡} is the Gibbs free energy of activation, k_{B} is Boltzmann's constant, and h is Planck's constant.

In another formulation, the contribution of individual reactions can be captured as a weighted sum of different functions representing different chemical components that contribute to the taste degradation:

TDR(T)=Σᵢ₌₁ⁿWᵢkᵢ(T) (Eq 6)

Where kᵢ(T) is the chemical reaction rate for a specific component as specified beforehand. One recognizes that in all these expressions the temperature T is a function of time.

For practical purposes, kᵢ(T) can be any of the functions mentioned previously. The coefficients in these equations can be determined either theoretically or can be chosen to fit experimental data. More generally, an empirically constructed curve can be used instead in which case an analytical expression can be obtained by a curve fit.

To render the computational process more efficient, different approximation can be used. One example is the Taylor series expansion allowing the use of only a few lower order terms

$$k ( T ) = k ( {T}_{0} ) + \frac{{k}^{′} ( {T}_{0} )}{1 !} ( T - {T}_{0} ) + \frac{{k}^{″} ( {T}_{0} )}{2 !} {( T - {T}_{0} )}^{2} + … HOT \qquad ( Eq 7 )$$

where HOT stands for “Higher Order Terms”.

Another practical solution is to consider a series expansion over small temperature intervals. This can produce, for example, a piecewise constant function as shown in FIG. 5.

Another method can set an objective to best match certain taste parameters. In this case an objective function can be constructed based on the difference between the predictions and the results to be matched. An optimization algorithm can be used to determine a “best” damage taste rate which would minimize the objective function. The dependence of the burn damage taste rate on temperature will be the design variable in this approach.

For the purpose of this analysis, the form (1) of the Arrhenius equation is considered. The coefficients are determined based on available values and practical observations.

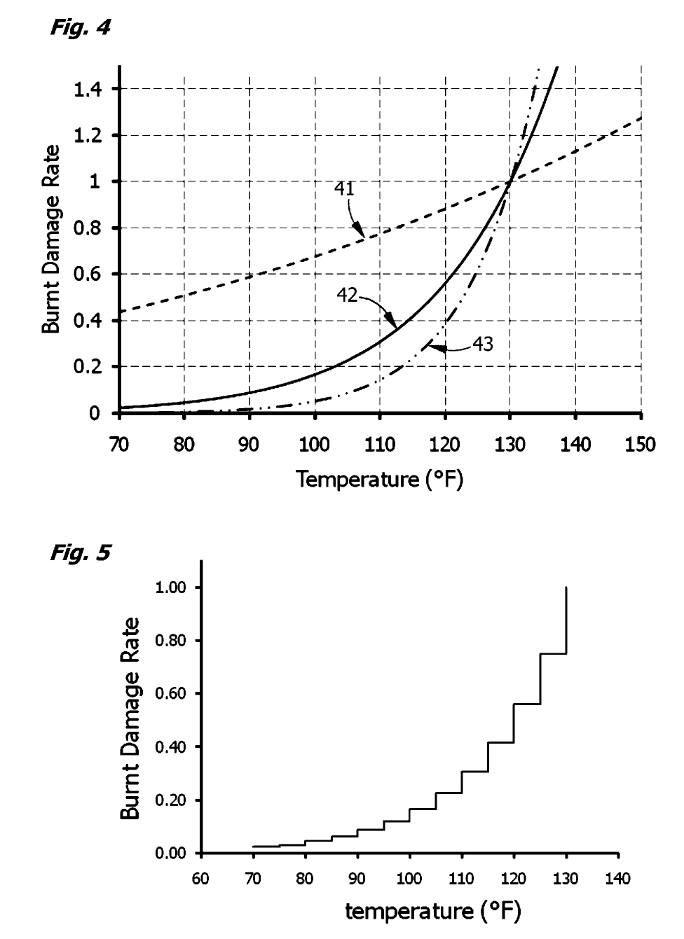

Typical values for the activation energy around room temperature run from 20 to 150 kJ/mol. With gas constant R=8.314 J/mol-° K, normal values for Eₐ/R that are between 0.24×10⁴ and 1.8×10⁴. The reaction rate variations with temperature for 20 kJ/mol (dashed line), 90 kJ/mol (solid line) and 150 kJ/mol (dashed-dotted line) for the activation energy are presented in FIG. 4. It has been observed that there is little depreciation in taste when coffee is at room temperature, say 70° F. At this temperature, the burn damage rate is therefore close to zero for all practical purposes. At 130° F., however, there is an accelerated depreciation in taste. A value of 1.0 will be assigned for the burn damage rate at 130° F. The constant A in equation 1 (Eq 1) can be determined to satisfy this condition. In order to pick a representative curve we look at FIG. 4. The dashed line (41) overestimates the burnt taste at room temperature as it is much larger than zero. The dashed-dotted line (43) underestimates the burnt taste at temperatures between 90° F. and 110° F. In between these curves, the solid line (42) agrees well with practical observations. It has a small reaction rate at room temperature and a large, rapidly increasing reaction rate at temperatures close or above 130° F. Therefore, a value of 1.1×10⁴ is considered for the Eₐ/R exponential factor in this analysis.

Since in this form the equation is specifically calibrated to determine the burnt taste, the result will then be called ‘Burn Damage Rate’ or simply ‘Burn Damage’ (BD). The equation becomes

$$BD ( T ) = \frac{1}{2.6134} * {10}^{15} * {e}^{- \frac{1.1 \times {10}^{4}}{T}} \qquad ( Eq 8 )$$

A total burn damage score (TBDS) at certain time t₁ is obtained by integrating the burn damage rate over the observation period started at t₀

TBDS(t₁)=∫ₜ₀^{t1}BD(T)dt (Eq. 9)

where the temperature T is a function of time.

TBDS can then be used to compare different heating and cooling patterns and to assess taste degradation over a period of time.

### Temperature History, TBDS, and Flavor

The method is best understood by the power of a few examples. To construct realistic temperature profiles a number of experiments for heating or cooling a real cup of coffee were performed. Curve fits were used to obtain intermediate data points for the analysis.

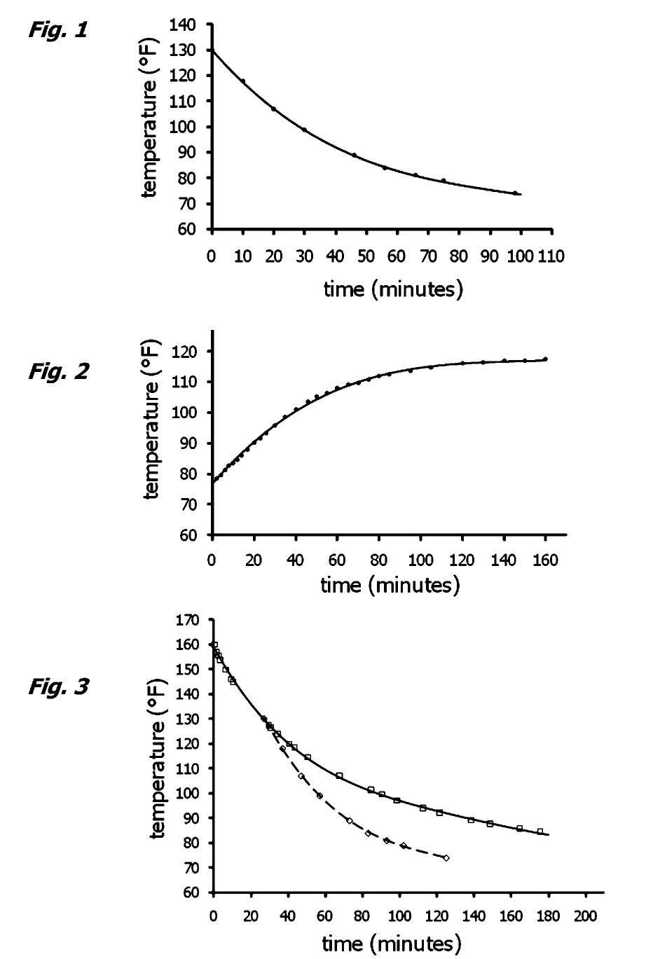

As would be expected, a liquid beverage cools by an exponential decay in temperature as shown in FIG. 1, which presents the cooling process of 8 oz of water in a ceramic cup. The cooling curve is steepest at higher temperatures and flattens out at lower temperatures.

FIG. 2 presents the warming curve for a cup of liquid when set on a 21 W desktop coffee warmer of the prior art. As an observation for the inadequacy of this method, the temperature in the cup achieved by the unit reaches a plateau below the generally accepted optimum for a satisfactory cup of coffee.

FIG. 3 presents the cooling of the same quantity of a liquid in a more thermally insulated travel mug with lid (solid line). For comparison, cooling of a ceramic mug is plotted on the same graph (dashed line). As one would expect, the two vessels produce different temperature profiles.

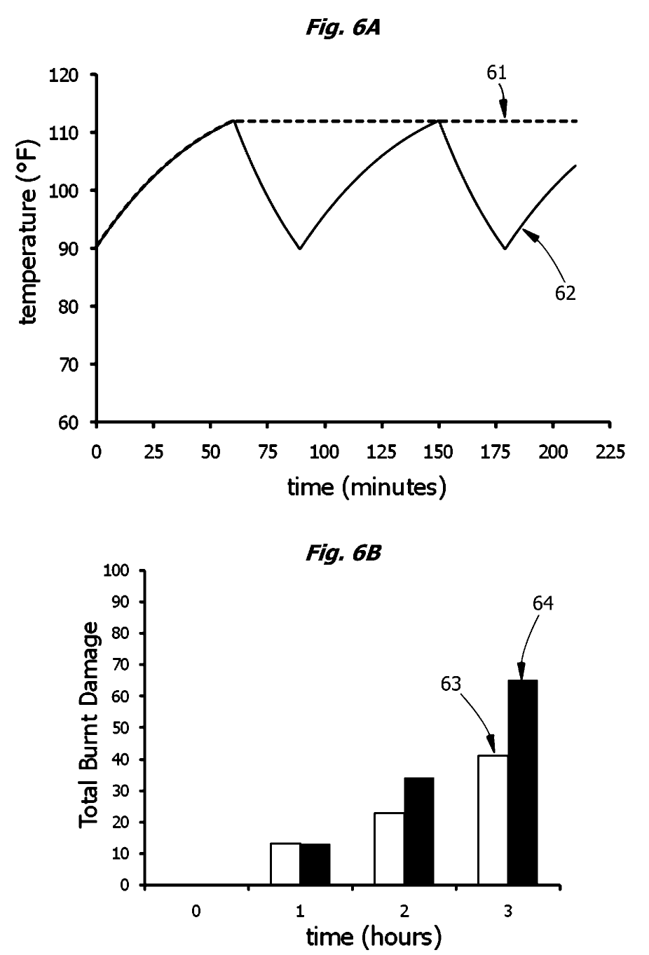

In a first application, a temperature profile for an 8 fl oz cup of coffee brewed at 90° F. and then brought to 112° F. is considered under two heating regimes. In the first regime (dotted line 61, FIG. 6A), the coffee is brought to constant temperature and held there. This can be accomplished with resistive heating and is representative of conventional art, such as the “Coffee Cup Warmer” cited earlier as commercially available. In the other regime (solid line, 62), coffee is repetitively cycled thru heating to 112° F. and natural cooling down to 90° F., forming a sawtooth wave (62) in the heating profile. The warm-up period of about an hour to arrive at 112° F. was determined experimentally using a commercially available device, and represents a plateau temperature which approaches the maximal temperature achievable by the device. Note that the coffee could not be brought to the preferred target temperature of between 125° F. and 155° F. Total burn damage scores (TBDS) are plotted in FIG. 6B. Over a 3 hr period, a TBDS score is obtained by integrating Eq. 8 over the temperature history. For the constant temperature regime (black bar, 64) the result was 65.4 versus 41.3 for the sawtooth heating profile (open bar, 63). It can be seen that by using a different heating method the taste has improved almost 60% at the end of 210 minutes of heating. This corresponds to a detectable difference in the burnt taste of coffee. While cooling coffee in order to periodically savor it hot would seem contrary to the teachings of the art as conventionally practiced, the model demonstrates a hitherto unrealized potential. A device designed to deliver periodic warming will be superior in preserving coffee flavor over a device designed for constant heating.

However, applying the sawtooth heating method to resistive heating is not practical due to low heat transfer rates which introduce a sluggish thermal response in the cup. This means that heat needs to be applied continuously over extended periods of time so that the beverage stays at a comfortable temperature.

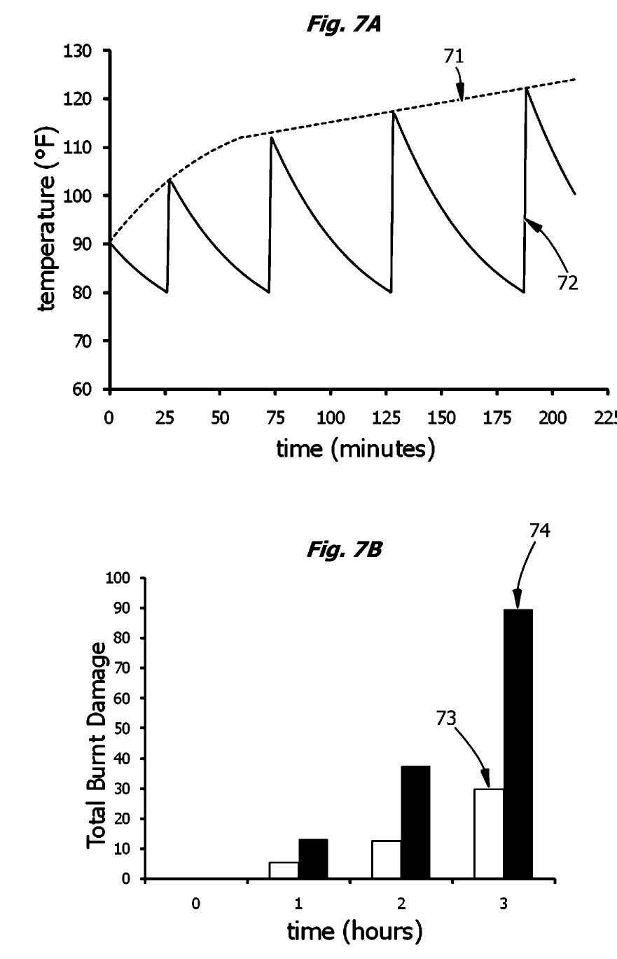

A second temperature history is illustrated in FIG. 7A. In this example the dotted line (71) represents a more realistic use of a constant resistive heating element without sensor control, so that the temperature progressively increases as the volume in the cup is consumed. The sawtooth wave (solid line 72, FIG. 7A) represents a device where heating is supplied periodically using a fast-acting method such as inductive heating. Because the response is quick, the heating can be applied precisely at the tasting time. By matching the sawtooth heating peaks to the resistive heating profile the comfort zones become equivalent.

The results are summarized in FIG. 7B, where it can be seen that continuous resistive heating (solid bar, 74) results in a TBD score of almost 90 over 3 hrs, corresponding to a very bitter taste, whereas a combination of periodic induction heating followed by a cooling period results in a substantially lower TBDS score (open bar, 73) of only 30. The accumulated burn damage is three times higher for the resistive continuous heating representative of existing cup warmers than for a novel induction heating coffee cup warmer that will match the temperature comfort of the resistive warmer. The final temperature in each case is about 124° F. even though inductive heating is perfectly capable of reaching higher temperatures while resistive heating is limited in this respect. The induction heating regime used for this example is for comparison purposes only and the invention is not limited thereto. The response time for an induction heating unit is very short allowing, for example, raising the temperature of 8 oz of liquid by 30° F. in about a minute if a 300 W unit is used.

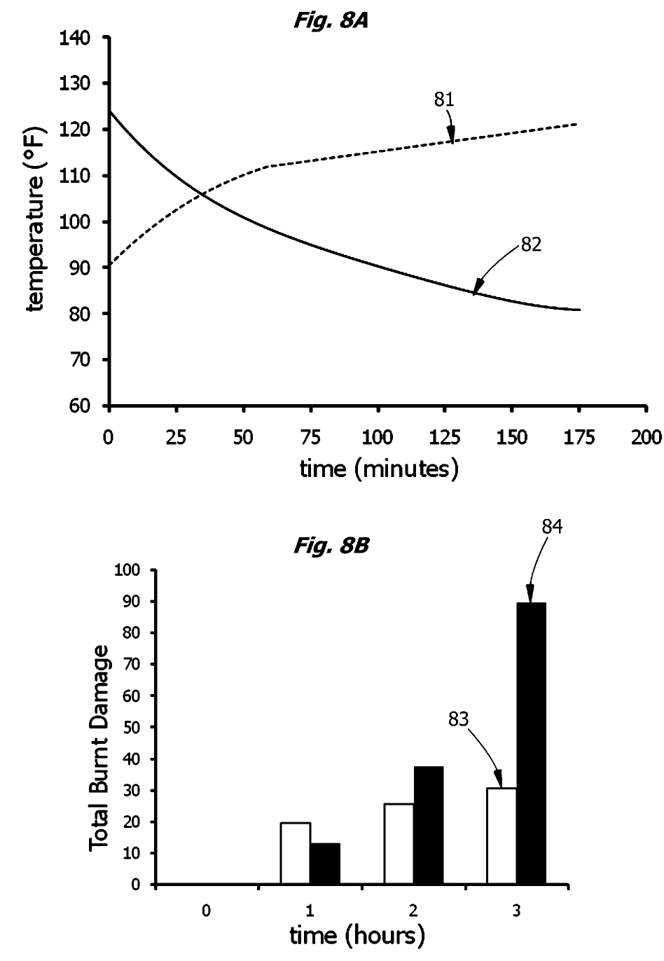

Another suggestive example is to compare the burn damage between a resistive pattern and a pattern characteristic of a thermally insulated traveling mug. The temperature histories for both are given in FIG. 8A. Here, the solid line (82) represents natural cooling in an insulated mug and the dotted line (81) represents a resistive heating temperature profile. Since temperatures are different along the observation period and there is no fast heating as in the case of induction heating, the temperature comfort cannot be matched. Nevertheless one can still compare the final burn damage.

FIG. 8B shows that the final burn damage after 3 hours in a thermal travel mug (open bar, 83) is three times lower than for the resistive heating (black bar, 84) and resembles (but is not equivalent since coffee temperature cannot be raised) induction heating (compare with FIG. 7B). Final burn damage after 3 hours (TBDS=29.1) stays low and in the same range as induction heating (FIG. 7B, TBDS=21.6) while being almost two and a half times lower than for the resistive heating (TBDS=70). This is consistent with current market trends where thermally insulated mugs or carafes tend to be favored over resistive heating. Even if the temperature in the thermally insulated vessel will constantly decrease, this is perceived as the better solution due to the disastrous depreciation in taste of the resistive method.

A better thermally insulated carafe can keep coffee warmer for longer periods of time but will also accumulate more burn damage. The present method of determining the burn taste damage can be use to compare different apparatus, such as carafes and travel mugs.

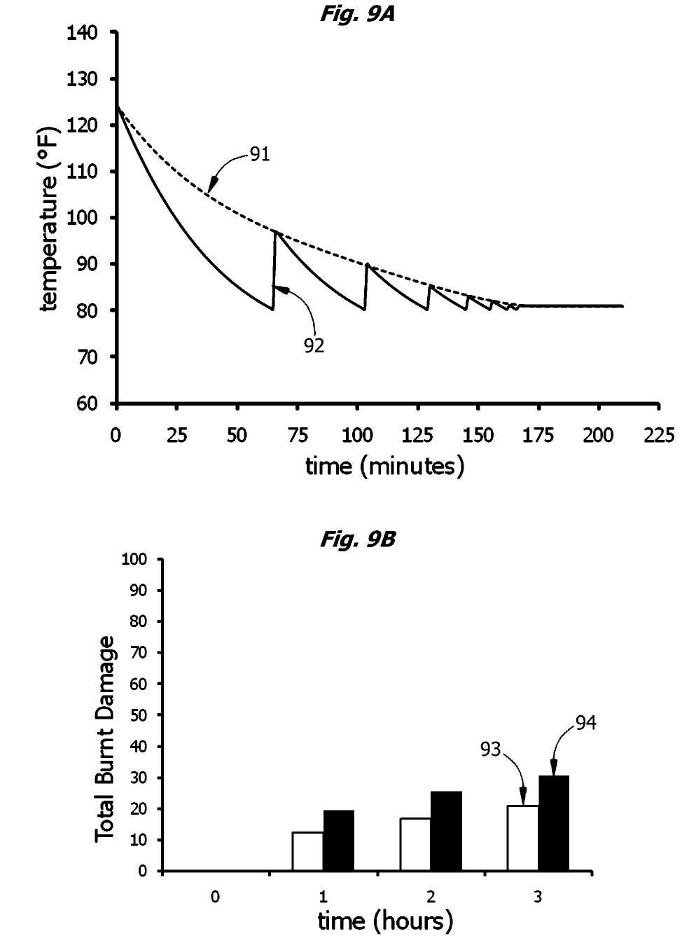

To directly compare induction heating with thermal insulation, the patterns in FIG. 9A have been considered, where the solid curve (92) represents intermittent induction heating and the dotted curve (91) represents the temperature profile associated with a thermally insulated mug. FIG. 9B shows that thermal insulation (black bar, 94) adds 50% more burn damage compared to induction heating (open bar, 93) at the same temperature comfort level. Induction heating has always outperformed all the other methods.

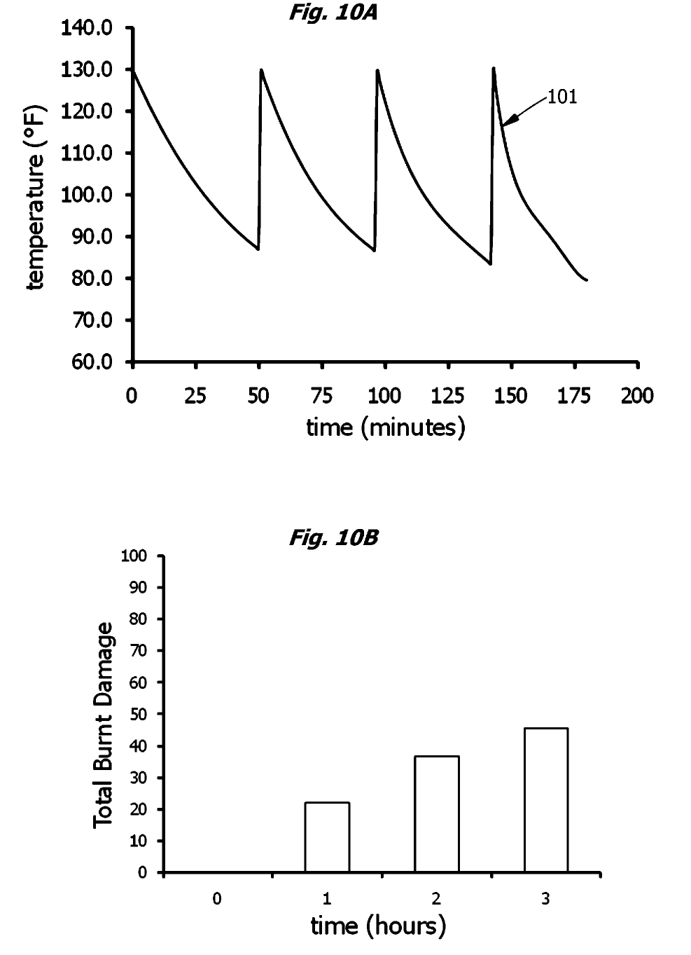

Turning now to FIG. 10A, the advantage of intermittent inductive heating is again demonstrated, where the time required for heating is much shorter and the coffee in the cup is heated only when desired hot by the user. A button is used to activate an inductive heating cycle every 45 minutes (solid line 101, FIG. 10A). Alternatively the coffee may be heated on demand with PID control so that the coffee is always reheated to the preferred target temperature zone, here 130° F. For simplicity, the assumption is made that the user chooses to sip 0.5 oz of coffee at intervals. Since a coffee cup may hold 5, 8, or even 12 fl oz of liquid, this rate of consumption could last for hours. Again to simplify comparisons, a 3 hr period is considered. It is assumed that the coffee is delivered freshly as brewed and preheated to 136° F. Since the coffee cools faster in smaller volume, different cooling curves have been used for each cooling leg. It is assumed that the heating always takes one minute.

The results for TBDS are presented as a bar graph in FIG. 10B. Final TBDS is only 45.7 at 3 hours even though the coffee is at greater than 110° F. more than half of the time. It is clearly shown that inductive heating (open bars) on demand is superior in reducing strain on coffee flavor caused by extended holding at elevated temperatures, i.e. the temperature history is reduced as compared to a constant heating regime. Since most coffee lovers do not sip coffee continuously, this pattern of heating is actually more closely in line with expected consumer practice.

These examples indicate that the prediction of the model correlates very well with taste observations.

By incorporating a “thermal burn damage monitor”—which includes a temperature sensor unit and functionality for on-board calculation of the burn damage rate and updating the total burn damage score, the consumer can be advised of the condition of the beverage and make a better decision about whether to consume it or seek a fresh cup.

The present analysis shows that the best method for making coffee or other hot beverages with similar properties available at a pleasant to drink temperature at times extended beyond its normal cooling time and with minimal degradation in taste is to let the beverage cool down once it is heated or brewed. When it needs to be reheated, heat is only applied immediately before consumption for manual systems or at prescribed time intervals or when a lower temperature is reached for automated systems. This is different than the current systems that keep coffee warm by continuously applying heat or by thermally insulating the vessel to slow down the cooling process.

It has also been shown that, for taste preservation, a fast heating method like induction heating or microwave oven heating is preferred over slow heating method as resistive heating of ceramic or glass cups or pots. Other beverages where taste might suffer from temperature variations can also benefit from this method.

The method finds application in assessing an accumulated burn taste in coffee and other hot beverages. A mathematical formula suitable for the rate of burn taste damage dependence with temperature is derived and the result then integrated over time to determine thermal burn taste damage (TBDS). Different heating patterns can then easily be compared for their effect on taste degradation by exposure to elevated temperatures. In a first embodiment, an apparatus for displaying this method is realized in a thermal burn damage monitor. This apparatus may be a stand-alone device or may be integrated into a carafe, travel mug, or percolator pot, for example.

The preferred embodiment would be a specialized heating unit for hot beverage like coffee in cups or pots that can be used at a convenient location. This heating pattern can be achieved by using a fixed or adjustable timer or a temperature sensing system that stops the heating when a certain temperature is reached. The heating can be restarted manually when warm beverage is desired or automatically when a prescribed lower temperature is reached or a certain amount of time has passed. For the latter case, the restart temperature needs to be significantly lower than the upper heating temperature in order to reduce burn taste.

A preferred embodiment that can satisfy the above requirements is an induction heating based system. Such units will require different functionality than existing portable induction heating units which are made to heat large vessels for long times. On the contrary, desktop coffee heaters need to heat small susceptors for relatively small periods of time. They also have to have a small profile to fit in crowded spaces, to make low or, better, no noise at all, which puts an important constraint on the unit cooling system or the components more likely to heat. They will also need to deliver enough power to heat the coffee fast enough for a “quick grab” and, at the same time, to keep the power level low enough so that they do not overstrain an outlet that might be already shared with other devices like computers, desk lamps, etc. It is considered that a power level below 100 W is inadequate to heat coffee fast enough. It will take about 4 minutes to heat 8 oz of coffee by 50° F. A maximum power limit is dictated more by the load put on the electrical system as well on the cooling restrictions and heating of internal components. An 800 W upper power limit is considered here even though the actual design may have less. This will allow heating 8 oz of coffee by 50° F. in less than one minute.

Another feature that differentiates the present desktop coffee cup inductive heating apparatus from exiting inductive heaters is the increased gap between the inductive coil and the susceptor. Ceramic cups and mugs have a concave bottom or have an extra bottom rim to reduce the thermal contact with the surface on which a mug is placed. Since the heating cartridge is placed inside the mug, the distance between the susceptor and the hob surface can be as much as ⅜ in, sometimes even more. This adds to the distance between the hob upper surface and the coil which can be ⅛ in or more. This means that the coil and the susceptor need to couple at a distance of about ½ in, sometimes more. Existing induction heating units do not heat at this gap distances. They usually do not heat vessels less than 4 inches in diameter, either.

Some exceptions may apply like in the case of induction heaters designed for use in vehicles. They may use mugs that do not have an elevated bottom rim and therefore the minimum required working gap may be reduced.

All these requirements are challenging enough to not have been pursued had it not been showed that this method of heating coffee is far superior to other existing methods.

Regular microwave ovens can also be used for coffee or tea heating. However, the currently available designs are relatively big and they are noisy. They are also more cumbersome to use since one has to open and close a door to insert the beverage container and then reopen and close again after reheating. Microwave ovens may not be used without a protective enclosure, a significant limitation in their application for desktop or car use.

Specialized resistive systems with a timer or temperature sensing system can also be used for heating coffee or tea especially for metal or bottom metal vessels that allow for better heat transfer and therefore shorter heating times. On the other hand, even though it is true that they heat faster, it is equally true metal vessels cool down faster too.

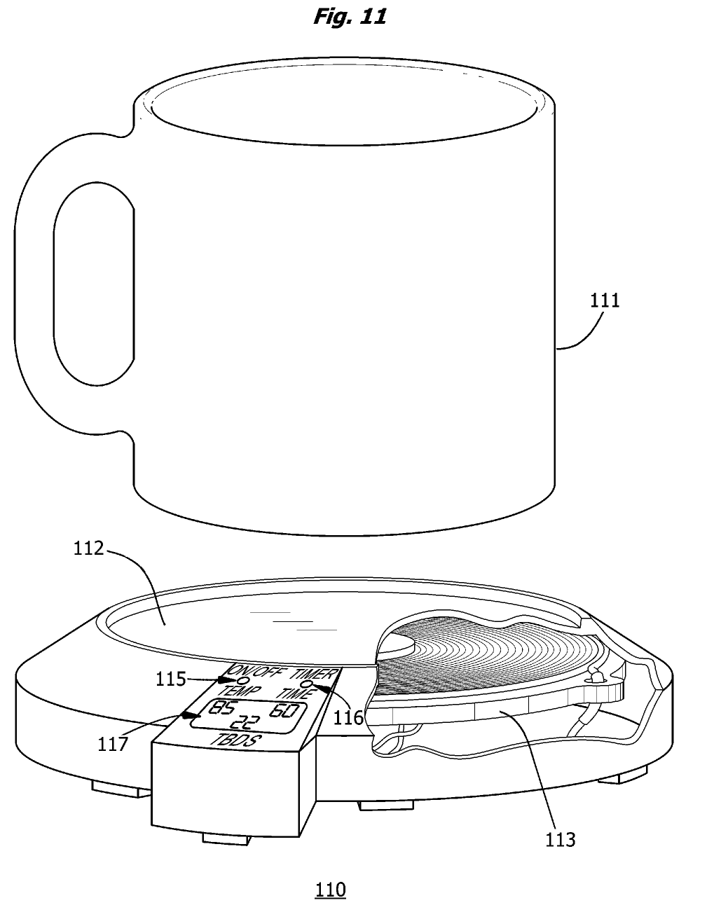

FIG. 11 shows an induction heating coffee cup warmer unit with an incremental timer. As shown for illustration, a warmer unit (110) is configured to support a cup (111) on a hob surface (112). Internal to the warmer unit is an inductive heating coil (113). Control surfaces include an ON/OFF button (115), a TIMER button (116) for incrementing a heating cycle, and a display (117), which may be configured to show current temperature, programmed heating time, and TBDS. The unit is designed to be placed on a desk or table and requires no enclosure to protect the user, as is necessary with a microwave heater.

For this preferred embodiment, the timer can be adjusted in increments of 10 seconds for up to one minute total heating time. Each time the TIMER button is pressed, the heating time is increased by 10 seconds. After the heating time becomes 60 seconds, the timer goes back to zero with the next press of the button and the setting continues in the same fashion. Once the timer is set for the desired time, the ON/OFF button or a separate START button turns on the unit. The heating will stop after the time set by the timer elapses. In this embodiment only one power setting for the heating unit is necessary.

Different heating times can be used to manually adjust for the quantity of coffee in the cup and for the desired drinking temperature. As an example, one can use 30 seconds to heat half a cup of coffee by 30° F. Or, one can use 40 seconds for heating the same amount of coffee that has stayed longer unused and become colder. To heat a full cup (8 oz) by 30° F. requires 60 seconds. If the final temperature is still not quite as desired, the coffee can be heated an additional 10 second by repeating the process. At the end of each heating cycle the timer resets itself to zero.

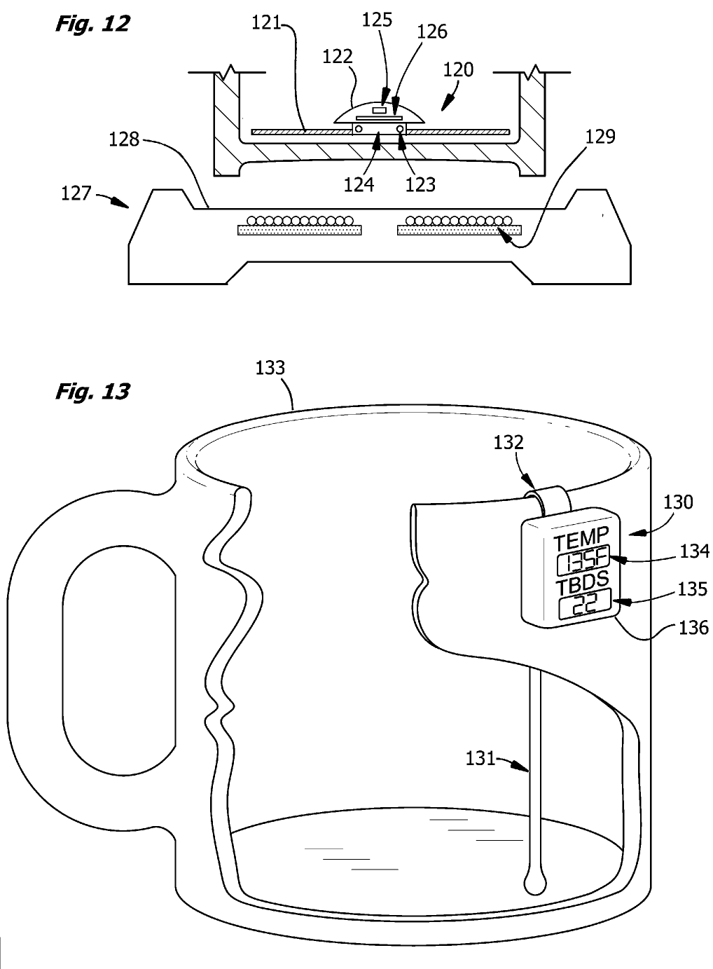

A similar goal can be achieved using a temperature sensor that transmits the coffee temperature to the induction heating unit. This can be done for example as shown here schematically in FIG. 12 in cross-section. The RFID tag is inserted into the cup while the receiver is in the unit itself. Shown is a schematic of a simplified inductive heating cartridge (120) with disk body (121) and centrally mounted RFID and sensor capsule assembly (122). The capsule assembly is mounted through the disk body and an RFID antenna (123) is located within the lower stem (124) of the capsule, in this embodiment. The RFID chip (126) and a temperature sensor (125) in electronic communication with the RFID chip circuitry are embedded in the capsule housing. Also shown in cutaway view is a desktop inductive heating unit (127) with hob (128), internal coil (129) and may include a control interface as described in U.S. patent application Ser. No. 12/493,077. The user may increase the temperature in increments or may set a desired target temperature.

As shown in FIG. 11, the user will set the desired heating temperature using for example +/−buttons and watch how the display changes. Once the temperature is set, the unit is started with the ON or START button. The unit will turn itself off once the coffee inside reaches the desired temperature. Alternatively, the heating can be activated using temperature increments similar to the time increments. Thus, one could program heating by 10° F., 20° F., up to, for instance, 50° F. by repeatedly pressing a button and then press START. For added safety, an RFID tag can be included so that the unit can check that the appropriate cartridge is used by receiving the information from the tag. If the handshake between the unit and the cartridge has not been established, the unit will not function. The heating unit may also be configured to display temperature and TBDS, where TBDS is calculated by a calculator functionality in the unit, the calculator comprising a microprocessor coupled to an internal clock, a memory for storing program instructions and data, and a power supply.

Turning now to FIG. 13, shown is a thermal history monitor device (130) with temperature probe (131) for automatically evaluating and displaying the taste of coffee based on its cumulative temperature history. A cutaway view of a mug demonstrates how the device is fixed with a clip (132) to the wall of the mug and the temperature probe is disposed at the bottom. The device includes displays for temperature (134) and TBDS (135). The cumulative thermal history of the liquid is monitored by the device and the total thermal burn damage is outputted incrementally to the TBDS display (135). The TBDS display may be calculated by one of a variety of formulae with instructions embedded in a memory provided inside the head of the device. The temperature probe may include or be coupled to an A/D converter as needed.

In this embodiment, the probe includes a compact body (136) with circuitry for assessing temperature via an electronic signal received from temperature probe (131). The circuitry is configured to calculate a thermal damage rate and increment a total burn damage score, and to display temperature (134) and TBDS (135) by conventional semiconductor means, for example. Controls such as an on-off switch and a reset switch may be provided for the user. The temperature probe may be a thermistor or a RTD.

A sliding clip may be provided to make the probe easily adjust to the depth of the cup. Other adjustable systems may be conceived for probe adjustability, such as spring systems.

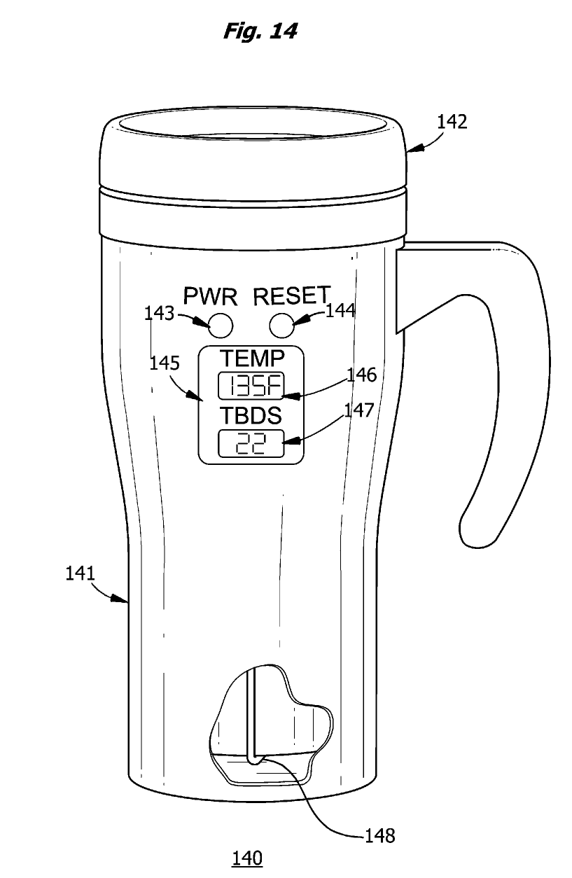

FIG. 14 illustrates an insulated travel mug with built-in temperature history evaluation and indicator device (140), where the display is based on a burn damage index algorithm of the invention and shows TBDS updated incrementally over time. The TBDS can be reported in a variety of ways besides the actual computational results. In one embodiment, the device display may be as simple as an LED that changes from green to red when the coffee has been exposed to an excessive duration of elevated temperature or an LCD (145) with fields for displaying temperature (146) and TBDS (147). In another embodiment the display (145) may be a countdown display that is initialized for example with a score or index of “100” and progressively counts down to zero as the quality of the coffee degrades. A bar system can also be adopted. Basic controls are also shown as representative of functionalities to be provided for the user. Shown are a reset button (144) and an on-off button (143) for conserving power. The temperature probe (148) is embedded in the inside wall and runs from the display circuitry to the base of the device, where the probe is in thermal contact with the liquid. The mug includes an insulated body (141) and lid (142).

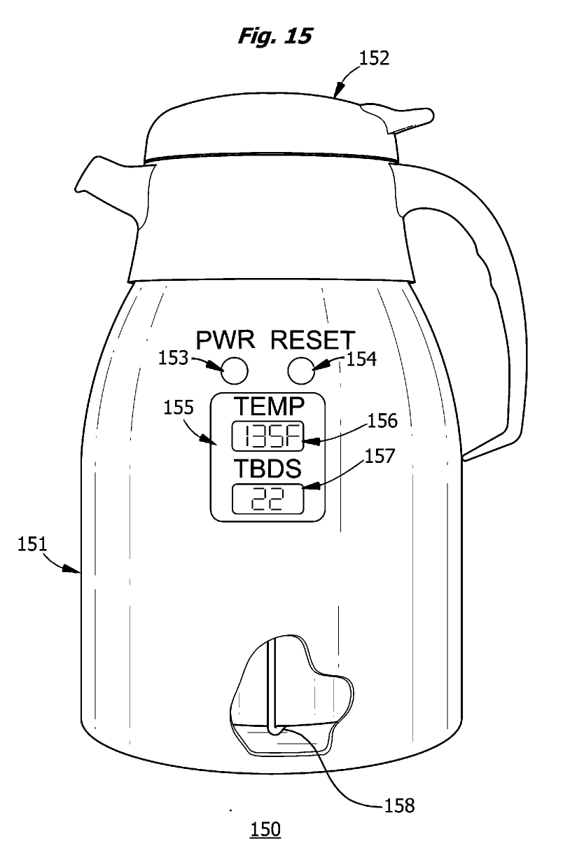

FIG. 15 is a conceptual view of a coffee carafe with integrated temperature history evaluation and indicator device (150) based on a burn damage index algorithm of the invention. The display (155) includes temperature (156) and TBDS (157). Also shown are controls for user convenience, including a reset button (153) and an on-off button (154). The display housing is gasketed to prevent water damage and shelters a battery. The temperature probe (158) is embedded and runs from the display circuitry to the inside base of the appliance, where the probe is in thermal contact with the liquid. The appliance includes an insulated body (151) and lid (152).

In devices of this kind, temperature sensor data is recorded with conventional RTD temperature probes, for example, and an algorithm is executed from firmware instructions or hardwired into a microprocessor, which is housed in a chip in the head of an insertable display probe of FIG. 13 or into the body of a vessel as shown in FIGS. 14-15. Semiconductor devices as known in the art are used to assess and update the accumulation of burn damage and the loss of desirable flavor and to display the results, for example using a small liquid crystal display, LED display, or simple colored lights. Each time a fresh cup is poured, or a fresh pot is made, the circuitry is reset and the process of assessing the thermal history begins again.

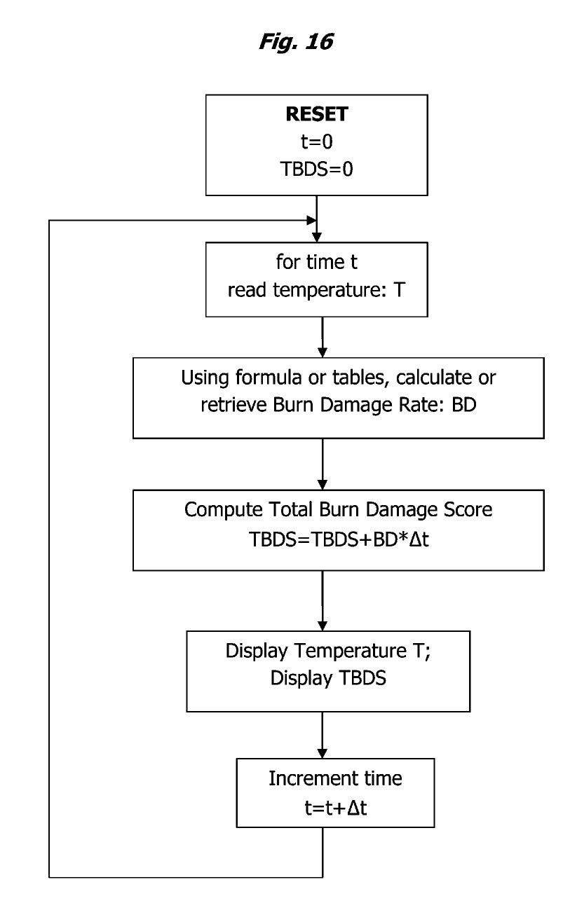

A simple logic circuit for updating a burn damage rate score in memory is shown schematically in FIG. 16. With the unit ON, pressing the RESET button on the unit initializes the counter or time t and TBDS parameters to zero. This is normally the time when coffee was just poured in the cup, but is chosen by the user. After a preset time increment Δt which is hardwired, the unit reads the temperature T from the probe. Based on the temperature reading, a Burn Damage Rate (BD) is calculated. The value can be obtained based on one of the formulas explained in the Specification or, for a piecewise constant formulation it can be retrieved from a table stored in the circuitry memory. As Δt is usually small, the integration is realized by adding the area under a constant BD variation, i.e. BD*Δt, to the cumulative TBDS as shown in FIG. 16. At certain convenient time intervals, usually larger than Δt, updated temperature and TBDS are displayed. After a new time increment passes, the process is repeated. Conditions may be established that the unit automatically shuts off after, for example, four hours, or if no temperatures variation is sensed, such as if the vessel is empty.

All the quantities, parameters, and control logic used in these examples are just for demonstrative purposes only. They can vary largely for different products.

### EXAMPLES

Comparative taste tests were conducted on coffee. By selecting suitable parameters for the formulae (Eqs 1-9), predicted TBDS was in good agreement with objective testing for flavor after exposure to thermal loads for up to 3 hrs. TBDS scores for different heating methods were inversely related to relative flavor quality.

### INCORPORATION BY REFERENCE

All of the U.S. patents, U.S. patent application publications, U.S. patent applications, foreign patents, foreign patent applications and non-patent publications referred to in this specification and related filings are incorporated herein by reference in their entirety.

While the above is a complete description of selected embodiments of the present invention, it is possible to practice the invention use various alternatives, modifications, combinations and equivalents. In general, in the following claims, the terms used in the written description should not be construed to limit the claims to specific specific embodiments described herein for illustration, but should be construed to include all possible embodiments, both specific and generic, along with the full scope of equivalents to which such claims are entitled. Accordingly, the claims are not limited by the disclosure.

I claim:

1. A method for assessing and comparing taste degradation for two methods of heating a coffee liquid in a vessel, which comprises
a) for a first method of heating said liquid:
i) measuring a temperature of said liquid at increments of time and calculating a first burn damage rate;
ii) summing said burn damage rate over said increments of time and reporting a first total burn damage score;
b) for a second method of heating said liquid:
i) measuring a temperature of said liquid at increments of time and calculating a second burn damage rate;
ii) summing said burn damage rate over said increments of time and reporting a second total burn damage score; and
c) comparing said first and said second total burn damage scores, the score indicating the degree of taste degradation.

2. The method of claim 1, further comprising displaying said first and said second total burn damage scores.

3. A method for assessing and displaying thermal burn damage to a potable liquid in a vessel, which comprises
a) a step for calculating a total burn damage score according to a mathematical function from a temperature profile as measured by a temperature probe in thermal contact with said liquid;
b) a step for incrementing a display to show the total burn damage score in real time to a user.

4. An apparatus for assessing and displaying thermal burn damage to a coffee liquid in a vessel, which comprises
a) a temperature probe;
b) an electronic circuit for reading a temperature of a liquid in said vessel, wherein said temperature probe is in thermal contact with said liquid; and
c) an electronic calculator comprising a microprocessor coupled to an internal clock and a memory, said calculator with a power supply;
d) a program used by said calculator that collects the temperature readings from the said temperature probe, solves for a burn damage rate from a first time to a second time separated by a time interval Δt, integrates said burn damage rate over said time interval Δt, and progressively increments the total burn damage score starting at a zero time; and
e) a display for displaying said incremented total burn damage score in real time to a user.

5. The apparatus of claim 4, wherein said vessel is a coffee cup and said electronic circuit and display are housed in a body, said body with clip for attachment to the outside wall of said coffee cup and with temperature probe, wherein said temperature probe is configured for immersion in said coffee liquid and for reporting said temperature to said electronic circuit.

6. The apparatus of claim 4, wherein said vessel is an insulated travel mug with body, base, walls and lid, wherein said electronic circuit, display and temperature probe are housed in said walls of said body, and further wherein said temperature probe is configured for extending into said base for making thermal contact with said coffee liquid in said vessel and for reporting said temperature to said electronic circuit.

7. The apparatus of claim 4, wherein said vessel is a coffee pot with walls, base and lid, wherein said electronic circuit, display and temperature probe are housed in said walls of said body, and further wherein said temperature probe is configured for extending into said base for making thermal contact with said coffee liquid in said vessel and for reporting said temperature to said electronic circuit.

8. The apparatus of claim 4, wherein said vessel is an airpot for coffee with walls, base and lid with pumpable spout, wherein said electronic circuit, display and temperature probe are housed in said walls of said body, and further wherein said temperature probe is configured for extending into said base for making thermal contact with said coffee liquid in said vessel and for reporting said temperature to said electronic circuit.

9. An apparatus for reheating a volume of a coffee liquid in a vessel, which comprises:
a) an open surface whereupon said vessel may rest;
b) an inductive coil under said open surface, said inductive coil having a power source of between 100 W and 800 W; and
c) a power circuit for energizing said inductive coil, wherein said power circuit is configured for on-demand intermittent heating of said coffee liquid in said vessel according to a cyclical method, said cyclical method comprising a step for heating said coffee liquid in response to a command from a user to a potable temperature and a step for immediately cooling said coffee liquid in a quiescent period thereafter, whereupon said user may repeat said heating step, whereby the coffee liquid in said vessel is heated with a temperature profile that follows a sawtooth curve.

10. The apparatus of claim 9, further comprising a temperature probe in thermal contact with said coffee liquid, said probe for measuring a temperature of said coffee liquid over increments of time and reporting said temperature to a calculator functionality, said calculator functionality comprising a microprocessor coupled to an internal clock and a memory for storing a program, said calculator having a power supply.

11. The apparatus of claim 10, wherein said cyclical method for on-demand heating further comprises
a) a step for calculating a total burn damage score as a function of said temperature profile measured by the apparatus;
b) a step for incrementing a display to show said total burn damage score in real time to said user; and
c) optionally, a step for controlling said power circuit to heat said coffee liquid according to said sawtooth curve so as to minimize the total burn damage score while providing said coffee liquid at said potable temperature when needed.

12. The apparatus according to claim 10, wherein a setpoint is chosen by said user to select said potable temperature according to an individual preference.
